(19) United States
(12) Patent Application Publication
(10) Pub. No.: US 2020/0163952 A1
(43) Pub. Date: May 28, 2020

(54) COMPOSITIONS AND METHODS FOR TREATING NERVE AGENT EXPOSURE

(71) Applicant: Steven Hoffman, Mahwah, NJ (US)

(72) Inventor: Steven Hoffman, Mahwah, NJ (US)

(21) Appl. No.: 16/695,821

(22) Filed: Nov. 26, 2019

Related U.S. Application Data
(60) Provisional application No. 62/771,460, filed on Nov. 26, 2018.

(51) Int. Cl. A61K 31/46; A61K 9/70; A61K 31/4425; A61K 47/26; A61K 47/10; A61K 47/20; A61K 47/18

(52) U.S. Cl. CPC A61K 31/46; A61K 9/7023; A61K 31/4425; A61K 45/06; A61K 47/10; A61K 47/20; A61K 47/186; A61K 47/26

(57) ABSTRACT

The present disclosure is directed to transdermal compositions comprising a poison antidote, and methods of using such compositions to counteract the effects of a poison.

### CROSS-REFERENCE TO RELATED APPLICATION

This application claims the benefit of priority to U.S. Provisional Application No. 62/771,460, filed Nov. 26, 2018, the entirety of which is incorporated by reference herein.

### BACKGROUND

Transdermal administration of therapeutic agents has many advantages, including convenience and avoidance of gastrointestinal tract metabolism. But in the absence of penetration enhancing agents, many therapeutic agents are not capable of penetrating the skin in therapeutically effective concentrations. As such, compositions that facilitate the penetration of therapeutic agents through the skin are needed.

A specific application in which transdermal delivery of a therapeutic agent is particularly useful is in the delivery of antidotes to poisons such as nerve agents. Oral dosage forms generally are not fast-acting enough to be useful under conditions of acute poisoning. Moreover, patients affected by poisons may not be able to successfully ingest such dosage forms. Patients affected by poisons also may lack the physical dexterity to self-administer antidotes by injection (e.g., intravenous or percutaneous administration). Transdermal administration, in contrast, provides an ideal mechanism by which to rapidly introduce antidote into the circulation under circumstances of acute poisoning.

Thus, there exists a need for transdermal compositions capable of delivering antidotes to patients suffering from acute poisoning, including in particular, patients suffering from exposure to nerve agents.

### SUMMARY

The present disclosure is directed to compositions comprising a poison antidote, a second component, a third component, a C₂₋₁₀alkyl alcohol, and an organic acid having 1 to 25 carbon atoms, wherein the poison antidote, and second and third components are further defined herein. Methods of making these compositions are also described, as are methods of using these compositions to counteract the effects of a poison.

### DETAILED DESCRIPTION OF ILLUSTRATIVE EMBODIMENTS

The present disclosure may be understood more readily by reference to the following detailed description of desired embodiments and the examples included therein. In the following specification and the claims that follow, reference will be made to a number of terms which have the following meanings.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, “alkyl” refers to straight chain and branched chains having the indicated number of carbon atoms, usually from 1 to 20 carbon atoms, for example 1 to 8 carbon atoms, such as 1 to 6 or 1 to 7 carbon atoms. For example C1-6 alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes n-propyl and isopropyl. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like.

As used herein, “alkenyl” refers to an unsaturated branched or straight-chain alkyl group having at least one carbon-carbon double bond. The group may be in either the cis or trans configuration about the double bond(s). The group may also be an aromatic group, for example, a phenyl or phenylene moiety. Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl; phenylene, and the like. In certain embodiments, an alkenyl group has from 2 to 20 carbon atoms.

As used herein, “alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

The present disclosure is directed to compositions that facilitate and/or enhance the transdermal permeation of a poison antidote through the skin. As used herein, the term “transdermal permeation” includes both percutaneous delivery and transmucosal delivery, that is, passage through skin or mucosal tissue and into the bloodstream. As used herein in reference to transdermal penetration, the term “enhancing” refers to increasing the rate at which a therapeutic agent penetrates the skin or mucosal tissue and enters the bloodstream. In some embodiments, these compositions include a poison antidote, a second component, a third component, an alcohol, an organic acid, and, optionally, water. Other compositions of the disclosure further comprise an anticonvulsant agent.

The compositions according to the disclosure comprise a poison antidote. The term “poison antidote,” as used herein, refers to any chemical substance that upon ingestion, inhalation, absorption, application, injection, or development within the body, counteracts the deleterious biological effects of a poison. As used herein, the term “poison” refers to any chemical substance that, upon ingestion, inhalation, absorption, application, injection, or development within the body, may cause injury, serious injury, or death. Examples of poisons include acetominofen (overdose), ethanol (overdose), anticholinesterases, methanol, arsenic, cyanide, benzodiazepine (overdose), ethylene glycol, lead, mercury, iron (overdose), folic acid antagonist (overdose), nerve agents, and opioids (overdose). In preferred aspects, the poison is a nerve agent and the poison antidote is a nerve agent antidote. The term “nerve agent,” as used herein, refers to any chemical substance that disrupts the mechanisms by which nerves transfer messages to organs in a human or some other mammal.

In some aspects of the present disclosure, the poison antidote is acetylcysteine (for acetominofen overdose), acetylcholinesterase (for nerve agent exposure), biperiden (for nerve agent exposure), butyrylcholinesterase (for nerve agent exposure), atropine (for nerve agent exposure), dimercaprol (for metal poisoning, e.g., arsenic, lead, mercury, iron), flumazenil (for benzodiazepine overdose), amyl nitrite (for cyanide poisoning), sodium nitrite/sodium thiosulfate (for cyanide poisoning), hydroxocobalamin (for cyanide poisoning), fomepizole (for ethylene glycol poisoning), leucovorin (for folic acid antagonist overdose), EDTA (for metal poisoning, e.g., arsenic, lead, mercury, iron), deferoxamine (for iron overdose), a pralidoxime salt (for nerve agent exposure), nalmefene (for opioid overdose), potassium 2,3-butanedione monoximate (for nerve agent exposure), 1-[[[4-(aminocarbonyl)-pyridinio]-methoxy]-methyl]-2-[(hydroxyimino)methyl]pyridinium dichloride (for nerve agent exposure), 2-[(hydroxyimino)methyl]-1-[4-(tert-butyl)benzyl]pyridinium bromide (for nerve agent exposure), or quaternary ammonium salts thereof, or pharmaceutically acceptable salts thereof, or mixtures thereof.

In some embodiments, the poison antidote is acetylcholinesterase (for nerve agent exposure), biperiden (for nerve agent exposure), butyrylcholinesterase (for nerve agent exposure), atropine (for nerve agent exposure), a pralidoxime salt (for nerve agent exposure), potassium 2,3-butanedione monoximate (for nerve agent exposure), 1-[[[4-(aminocarbonyl)-pyridinio]-methoxy]-methyl]-2-[(hydroxyimino) methyl] pyridinium dichloride (for nerve agent exposure), 2-[(hydroxyimino)methyl]-1-[4-(tert-butyl)benzyl]pyridinium bromide (for nerve agent exposure), or quaternary ammonium salts thereof, or pharmaceutically acceptable salts thereof, or mixtures thereof.

In some embodiments, the poison antidote is a pralidoxime salt. In some embodiments wherein the poison antidote is a pralidoxime salt, the pralidoxime salt is pralidoxime chloride (for nerve agent exposure), pralidoxime bromide (for nerve agent exposure), or pralidoxime iodide (for nerve agent exposure).

In some embodiments, the poison antidote is atropine or a pharmaceutically acceptable salt thereof. In yet other embodiments, the poison antidote is a mixture of atropine or pharmaceutically acceptable salt thereof, and a pralidoxime salt. In other embodiments, the poison antidote is a mixture of atropine or pharmaceutically acceptable salt thereof, and pralidoxime chloride, pralidoxime iodide, or pralidoxime bromide. In some embodiments, the poison antidote is a mixture of pralidoxime chloride or pralidoxime iodide and atropine.

The amount of poison antidote in the compositions according to the disclosure will be such that administration of the composition to the mammal's skin will result in an antidote concentration in the mammal's bloodstream that is therapeutically effective in counteracting the poison to which the antidote is directed. Those skilled in the art will be able to determine the amount of antidote to include in a given composition by taking into account, for example, the characteristics of the mammal's skin, the skin permeability of the poison antidote in the context of the composition, and the required systemic poison antidote concentration to counteract the effects of a given poison.

The compositions of the disclosure can comprise from about 0.01 vol. % to about 99 vol. % of the poison antidote. The compositions of the disclosure can comprise from about 0.01 vol. % to about 75 vol. % of the poison antidote. The compositions of the disclosure can comprise from about 0.01 vol. % to about 50 vol. % of the poison antidote. The compositions of the disclosure can comprise from about 0.01 vol. % to about 40 vol. % of the poison antidote. The compositions of the disclosure can comprise from about 0.01 vol. % to about 30 vol. % of the poison antidote. The compositions of the disclosure can comprise from about 0.01 vol. % to about 20 vol. % of the poison antidote. In some embodiments, the compositions comprise from about 0.01 vol % to about 15 vol % of the poison antidote. In preferred embodiments, the compositions comprise from about 0.01 vol. % to about 5 vol. % of the poison antidote. In other embodiments, the compositions comprise from about 0.01 vol. % to about 3 vol. % of the poison antidote. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 vol. % of the poison antidote.

According to the disclosure, the second component comprises one or more of the following:

a) a compound of formula I

R—(OCH₂CH₂)_{y}—OH (I)
wherein R is C₁₋₂₀alkyl, C₂₋₂₀alkenyl; or C₂₋₂₀alkynyl; and y is 1 to 25; or

b) a tetrafunctional block copolymer surfactant terminating in primary hydroxyl groups; or

c) a sorbitan derivative; or

d) a C₈₋₁₀alkyl ammonium salt; or

e) a compound of formula II

HO—(CH₂CH₂O)ₘ—C(CH₃)(C₄H₉)—C≡C—C(CH₃)(C₄H₉)—(OCH₂CH₂)ₙ—OH (II)
wherein m and n are each independently 1 to 25; or

f) a combination thereof.

In preferred embodiments of the disclosure, the second component is a compound of formula I. In some embodiments, R is C₁₋₂₀alkyl, which can either be a straight chain or branched alkyl. Preferred compounds of formula I wherein R is C₁₋₂₀alkyl include, for example, is cetomacrogol 1000; octadecan-1-ol, ethoxylated; polyoxyethylene(12)tridecyl ether; polyoxyethylene(10)tridecyl ether; fatty alcohol polyoxyethylene ether, polyoxyethylene branched nonylcyclohexyl ether (TRITON N-101), nonaethylene glycol monododecyl ether, 23-{[4-(2,4,4-trimethyl-2-pentanyl)cyclohexyl]oxy}-3,6,9,12,15,18,21-heptaoxatricosan-1-ol, and combinations thereof. Nonaethylene glycol monododecyl ether is particularly preferred.

In other embodiments, R is C₂₋₂₀alkenyl, which can either be a straight chain or branched alkenyl. Preferred compounds of formula I wherein R is C₂₋₂₀alkenyl include, for example, polyoxyl(10)oleyl ether, polyethylene glycol tert-octylphenyl ether (TRITON X-100), and combinations thereof.

In yet other embodiment, R is C₂₋₂₀alkynyl, which can either be a straight chain or branch alkynyl.

In those embodiments wherein the second component is a compound of formula I, y is 1 to 25. In preferred embodiments, y is 5 to 15, preferably 8 to 10, with 9 being particularly preferred. In other embodiments, y is 1, 2, 3, 4, 5, 6, 7, 8, 9, 10, 11, 12, 13, 14, 15, 16, 17, 18, 19, 20, 21, 22, 23, 24, or 25.

In other aspects of the disclosure, the second component is a tetrafunctional block copolymer surfactant terminating in primary hydroxyl groups. Such compounds are commercially available under the tradename TETRONIC and include ethylenediaminetetrakis(ethoxylate-Block-propoxylate).

In other embodiments of the discosure, the second component is a sorbitan derivative, for example, polyoxyethylene sorbitan tetraoleate, 1,4-anhydro-6-O-palmitoyl-D-glucitol (sorbitan, monohexadecanoate), a polyethylene glycol sorbitan monolaurate (e.g., TWEEN 20, TWEEN 40, TWEEN 60, TWEEN 85), and combinations thereof.

In still other embodiments of the disclosure, the second component is a C₈₋₁₀alkyl ammonium salt, for example, methyltrialkyl(C₈-C₁₀)ammonium chloride (ADOGEN 464).

In other embodiments, the second component is a compound of formula II.

The compositions of the disclosure can comprise from about 0.1 vol. % to about 40 vol. % of the second component. In preferred embodiments, the compositions comprise from about 1 vol. % to about 40 vol. % of the second component. In other embodiments, the compositions comprise from about 0.1 vol. % to about 5 vol. % of the second component. For example, the compositions can comprise about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 vol. % of the second component.

According to the disclosure, the compositions also include a third component that comprises one or more of the following:

a) an compound of the formula III

R²—N(R¹)—C(O)—R³ (III)
wherein
each R¹ is independently H or C₁₋₃alkyl; and
R² and R³ are independently C₁₋₇alkyl or together with the atoms to which they are attached, form a lactam having 3 to 10 carbon atoms; or

b) a sulfoxide; or

c) a urea; or

d) ethyl acetate; or

e) a combination thereof.

In preferred embodiments, the third component is compound of formula III. In some embodiments, R¹ is H. In other embodiments, R¹ is methyl, ethyl, propyl, or isopropyl, with methyl being particularly preferred. In some embodiments, R¹ is methyl, ethyl, or propyl.

In those embodiments wherein R² and R³ are independently C₁₋₇alkyl, each of R² and R³ is independently methyl, ethyl, propyl, isopropyl, butyl, s-butyl, t-butyl, pentyl, hexyl, or heptyl.

Preferably, R² and R³, together with the atoms to which they are attached, form a lactam having 3 to 10 carbon atoms. For example, the lactam can include 3, 4, 5, 6, 7, 8, 9, or 10 carbons, which can be a part of the lactam ring or which can form exocyclic branching. Examples of preferred lactams include pyrrolidones such as 2-pyrrolidone, 1-methyl-2-pyrrolidone, 5-methyl-2-pyrrolidone, and 1-ethyl-2-pyrrolidone. Preferably, the lactam is 1-methyl-2-pyrrolidinone or 2-pyrrolidone.

In some embodiments, the third component is a sulfoxide, for example, dimethyl sulfoxide.

In other embodiments, the third component is a urea, for example an imidazolidinone.

The compositions of the disclosure can comprise from about 0.01 vol. % to about 10 vol. % of the third component. In preferred embodiments, the compositions comprise from about 0.01 vol. % to about 5 vol. % of the third component. In other embodiments, the compositions comprise from about 0.01 vol. % to about 4 vol. % of the third component. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or about 10 vol. % of the third component.

In some embodiments of the disclosure, the ratio, by volume, of the second component to the third component is about 10:1 to about 4:1.

Alcohols for use in the compositions of the disclosure include C₂₋₁₀alkyl alcohols having at least one —OH moiety or at least two —OH moieties. For example, preferred alcohols include glycerol, propylene glycol, ethanol, isopropanol, 1-propanol, butanol, t-butanol, pentanol, 1-octanol, and combinations thereof, with ethanol being particularly preferred.

The compositions of the disclosure can comprise from about 0.1 vol. % to about 50 vol. % of the C₂₋₁₀alkyl alcohol. In preferred embodiments, the compositions comprise from about 1 vol. % to about 50 vol. % of the C₂₋₁₀ alkyl alcohol. In other embodiments, the compositions comprise from about 0.1 vol. % to about 5 vol. % of the C₂₋₁₀ alkyl alcohol. For example, the compositions can comprise about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 vol. % of the C₂₋₁₀ alkyl alcohol.

The compositions of the disclosure also include an organic acid having 1 to 25 carbon atoms. For example, organic acids for use in the disclose compositions include acetic acid, ascorbic acid, lactic acid, glycolic acid, propionic acid, and combinations thereof.

Other organic acids for use in the disclosure include fatty acids. As used herein, the term “fatty acid” has its ordinary meaning as would be understood by a person of ordinary skill in the art and includes a molecule having a carboxylic group and a hydrocarbon chain. Descriptions of the number of carbon atoms in a fatty acid herein refer to the number of carbon atoms in the hydrocarbon chain of the fatty acid, irrespective of whether the hydrocarbon chain is straight or branched. In some embodiments, the organic acid is a fatty acid or a C₁₋₆alkyl acid.

As used herein, the term “fatty acid” includes saturated fatty acids, which do not contain any double or triple bonds in the hydrocarbon chain. Saturated fatty acids include, but are not limited to propionic acid (C3) (by way of example, C3 indicates propionic acid has 3 carbon atoms in its hydrocarbon chain; the number of carbon atoms in the hydrocarbon chain of other example fatty acids is denoted in analogous fashion herein), butyric acid (C4), valeric acid (C5), caproic acid (C6), enanthic acid (C7), caprylic acid (C8), pelargonic acid (C9), capric acid (C10), undecylic acid (C11), lauric acid (C12), tridecylic acid (C13), myristic acid (C14), pentadecylic acid (C15), palmitic acid (C16), margaric acid (C17), stearic acid (C18), isostearic acid (C18), nonadecylic acid (C19), arachidic acid (C20), heneicosylic acid (C21), behenic acid (C22), tricosylic acid (C23), lignoceric acid (C24), pentacosylic acid (C25), cerotic acid (C26), heptacosylic acid (C27), montanic acid (C28), nonacocylic acid (C29), melissic acid (C30), henatriacontylic acid (C31), lacceroic acid (C32), psyllic acid (C33), geddic acid (C34), ceroplastic acid (C35) and hexatriacontylic acid (C36).

As used herein, the term “fatty acid” also includes monounsaturated fatty acids, which contain one double or triple bond in the hydrocarbon chain, and polyunsaturated fatty acids, which contain more than one double and/or triple bond in the hydrocarbon chain. Such acids include, but are not limited to the omega 3, omega 6, omega 9 fatty acids, other fatty acids such as myristoleic and palmitoleic acid and conjugated fatty acids. Examples of monounsaturated and polyunsaturated fatty acids include but are not limited to, (a) omega 3 fatty acids, such as hexadecatrienoic acid (C16:3); (by way of example, C16:3 indicates hexadecatrienoic acid has 16 carbon atoms in its hydrocarbon chain and 3 double bonds; the number of carbon atoms and double bonds in the hydrocarbon chain of other example unsaturated fatty acids is denoted in analogous fashion herein), alpha linolenic acid (C18:3) and eicosapentanoic acid (20:5), (b) omega 6 fatty acids, such as linoleic acid (18:2), docosadienoic acid (C22:2), arachidonic acid (C20:4) and tetracosatetraenoic acid (C24:5), (c) omega 9 fatty acids, such as oleic acid (C18:1), eicosenoic acid (C20:1) and nevronic acid (C24:1), and (d) conjugated fatty acids such as rumenic acid (C18:2), eleostatic acid (C18:3), and rumelenic acid (C18:3).

As used herein, the term “fatty acid” also includes branched fatty acids. Examples of branched fatty acids include, but are not limited to, monomethyl branched fatty acids, such as 14-methyl pentadecanoic acid, 6-methyl caprylic acid, 4-methyl-3-pentenoic acid, (pyroterebic acid), 2-methyl-2E-butenoic acid (tiglic acid), 2-methyl-2Z-butenoic acid (angelic acid), multimethyl branched acids, isoprenoid fatty acids (vittatalactone, all-trans-retinoic acid), branched methoxy fatty acids and hydroxy and other fatty acids such as 2-hydroxyoctanoic acid and 4-oxopentanoic acid.

The compositions of the disclosure can comprise from about 0.01 vol. % to about 15 vol. % of the organic acid. In some embodiment, the compositions comprise from about 1 vol % to about 15 vol % of the organic acid. In preferred embodiments, the compositions comprise from about 0.01 vol. % to about 5 vol. % of the organic acid. In other embodiments, the compositions comprise from about 0.01 vol. % to about 3 vol. % of the organic acid. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 vol. % of the organic acid.

Compositions of the disclosure can be anhydrous. As used herein, “anhydrous” refers to compositions comprising less than 1 vol. % of water, preferably less than 0.05 vol. % or less than 0.025 vol. % of water. Methods of determining water content are known in the art.

Compositions of the disclosure can include water. In some embodiments, the compositions can comprise up to 99 vol. % of water. In still other aspects, the compositions can comprise 5, 10, 20, 30, 40, 50, 60, 70, 80, 90, 95, or 99 vol. % of water. In other embodiments, the compositions can comprise 90, 91, 92, 93, 94, 95, 96, 97, 98, or 99 vol. % of water.

Compositions of the disclosure that include water can optionally contain one or more physiologically acceptable salts. While not being bound by any particular theory, it is believed that controlling the amount of salt that is present allows one to control the depth to which the present composition penetrates skin, with the concentration of salt having a generally inverse relationship to the penetration depth. Salts for use in the compositions include, but are not limited to, sodium chloride, potassium chloride, and mixtures thereof. A preferred form of sodium chloride is bacteriostatic sodium chloride solution.

The compositions of the disclosure may also encompass an anticonvulsant. Nerve agent exposure often induces convulsions that arise from a buildup of acetylcholine and resulting hyperstimulation of the cholinergic system. Many different classes of anticonvulsant may be used in the compositions of the present disclosure, including aldehydes, aromatic allylic alcohols, benzodiazepines, barbiturates, bromides, carbamates, carboxamides, fatty acids, GABA analogs hydantoins, oxazolidinediones propionates, pyrimidinediones, pyrrolidines, succinimides, sulfonamides, triazines, ureas, and valproylamides. In some embodiments, the anticonvulsant is acetazolamide, acetazolamide, barbexaclone, beclamide, brivaracetam, carbamazepine, clobazam, clonazepam, clorazepate, diazepam, divalproex sodium, eslicarbazepine, ethadione, ethosuximide, ethotoin, felbamate, fosphenytoin, gabapentin, lacosamide, lamotrigine, levitiracetam, lorazepam, mephenytoin, mesuximide, metharbital, methazolamide, methsuximide, methylphenobarbital, midazolam, nimetazepam, nitrazepam, oxcarbazepine, paraldehyde, paramethadione, perampanel, phenacemide, pheneturide, phenobarbital, phensuximide, phenytoin, potassium bromide, pregabalin, primidone, progabide, scopolamine, seletracetam, sodium valproate, stiripentol, sultiame, temazepam, tiagabine, topiramate, trimethadione, valnoctamide, valproic acid, valpromide, vigabatrin, or zonisamide. In some embodiments, the anticonvulsant is diazepam, midazolam, or scopolamine. In some embodiments, the anticonvulsant is diazepam or midazolam. In some embodiments, the anticonvulsant is diazepam. In other embodiments, the anticonvulsant is midazolam.

Compositions of the invention may be designed to be administered to the skin or mucosal tissue of a patient in need of treatment. Compositions of the invention may be formulated as gels, transdermal patches, lotions, creams, sprays, mists, emulsions, or dispersions. Appropriate excipients for formulating a gel, transdermal patch, lotion, cream, spray, or mist are readily apparent to a person of skill in the art and include, but are not limited to, stabilizers, emulsifiers, thickeners, antimicrobials, humectants, propellants, spreading agents, polymers, and adhesives, such as pressure sensitive adhesives. In particular, excipients that may be used to form a transdermal gel include, but are not limited to, alcohols, glycols, glycerin, butylated hydroxytoluene (BHT), and water.

The compositions of the disclosure will, in some embodiments, include, in addition to the above-discussed components, one or more additional components. Additional components include, but are not limited to, a transdermal absorption enhancer, a preservative (e.g., paraben), an antioxidant, a stabilizing agent, a filling agent that contains a hydrophilic polymer; a cross-linking agents; and a plasticizing agent.

In some embodiments, the compositions of the disclosure comprise about 2 vol % poison antidote; about 34 vol. % of the second component; about 4 vol. % of the third component; about 48 vol. % of the C₂₋₁₀alkyl alcohol; and about 12 vol. % of the organic acid. In other embodiments, the compositions of the disclosure comprise about 2 vol. % poison antidote; about 5 vol. % of the second component; about 0.5 vol. % of the third component; about 7 vol. % of the C₂₋₁₀alkyl alcohol; about 2 vol. % of the organic acid; and about 83 vol. % of the water.

The compositions of the disclosure can be used to administer a poison antidote to a mammal. For example, in preferred embodiments, these methods comprise applying any of the described compositions to the skin of a mammal for a time sufficient to achieve permeation of at least a portion of the poison antidote through the skin. Poison antidote skin permeation can be measured using techniques known in the art. In some embodiments, the mammal is a human being.

The compositions described herein can be applied to any convenient skin surface. Skin surfaces of interest include, but are not limited to: arms, leg, torso, head, neck, etc. The surface area that is covered by the transdermal formulation following application is generally sufficient to provide for the desired amount of poison antidote administration, and in certain embodiments ranges from about 1 cm² to about 200 cm².

The compositions described herein can be applied a single time or a plurality of times over a given time period, e.g., the course of the condition being treated, where the dosing schedule when a plurality of patches are administered over a given time period may be daily, weekly, biweekly, monthly, etc.

Also within the scope of the disclosure are methods comprising administering any of the described compositions to the skin of a mammal for a time and under conditions effective to achieve passage of at least a portion of the composition through the skin. Skin permeation can be measured using techniques known in the art.

In some aspects, the disclosure is directed to a method of counteracting the effects of a poison in a patient in need thereof, comprising administering to the skin of said patient an effective amount of a composition of the present disclosure. In this context, “effective amount” means an amount of composition that results in a systemic concentration of poison antidote that is effective to counteract the effects of the poison. Thus, an “effective amount” is an amount sufficient to ameliorate at least one of the effects of the poison.

In some embodiments, the poison is a nerve agent. Thus, in some aspects, the disclosure is directed to a method of counteracting the effects of a nerve agent in a patient in need thereof, comprising administering to the skin of said patient an effective amount of a composition of the present disclosure. A nerve agent, generally speaking, is a chemical compound that disrupts the normal functioning of the nervous system. Without intending to be bound by theory, a nerve agent inhibits acetylcholinesterase and thereby may cause the deleterious accumulation of neurotransmitters such as acetylcholine. In this context, an “effective amount” means an amount of composition that is effective to ameliorate one or more of the effects of the nerve agent, which include excessive production of mucous, tears, saliva and sweat; headache; stomach pain, nausea and vomiting; chest tightness and shortness of breath; loss of bladder and bowel control; muscle twitching; violent muscle spasms; seizures; coma; or death.

A person of ordinary skill in the art will be aware of several classes of nerve agents, including G-series nerve agents, V-series nerve agents, Novichok agents, and carbamates.

In some embodiments, the disclosure is directed to a method of counteracting the effects of a G-series nerve agent in a patient in need thereof, comprising administering to the skin of said patient an effective amount of a composition of the present disclosure. In some embodiments, the G-series nerve agent is ethyl N,N-dimethylphosphoramidocyanidate (tabun, GA), propan-2-yl methylphosphonofluoridate (sarin, GB), O-pinacolyl methylphosphonofluoridate (soman, GD), cyclohexyl methylphosphonofluoridate (cyclosarin, GF), or 2-(Dimethylamino)ethyl N,N-dimethylphosphoramidofluoridate) (GV).

In some embodiments, the disclosure is directed to a method of counteracting the effects of a V-series nerve agent in a patient in need thereof, comprising administering to the skin of said patient an effective amount of a composition of the present disclosure. In some embodiments, the V-series nerve agent is (S)-(ethyl {[2-(diethylamino)ethyl]sulfanyl}(ethyl)phosphinate) (VE), O,O-Diethyl S[2-(diethylamino)ethyl]phosphorothioate (VG), S[2-(Diethylamino)ethyl] O-ethyl methylphosphonothioate (VM), N,N-diethyl-2-(methyl-(2-methylpropoxy)phosphoryl)sulfanylethanamine (VR), Ethyl ({2-[bis(propan-2-yl)amino]ethyl}sulfanyl)(methyl)phosphinate (VX), or O-cyclopentyl S-(2-diethylaminoethyl) methylphosphonothiolate (EA-3148).

In some embodiments, the disclosure is directed to a method of counteracting the effects of a Novichok nerve agent in a patient in need thereof, comprising administering to the skin of said patient an effective amount of a composition of the present disclosure. Novichok nerve agents are a series of nerve agents developed in the former Soviet Union and Russia. In some embodiments, the Novichok nerve agent is ([(2-chloro-1-methylpropoxy)fluorohydroxyphosphinyl]oxy)carbonimidic chloride fluoride, (E)-N-(1-(diethylamino)ethylidene)-P-methylphosphonamidic fluoride, 1-chloropropan-2-yl (E)-(((chlorofluoromethylene)amino)oxy)phosphonofluoridate, 2-(dimethylamino)ethyl ethyl dimethylphosphoramidate, ethyl (bis(diethylamino)methylene)phosphoramidofluoridate, ethyl (E)-(1-(diethylamino)ethylidene)phosphoramidofluoridate, ethyl dimethylphosphoramidofluoridate, ethyl N-[(1E)-1-(diethylamino)ethylidene]-phosphoramidofluoridate, methyl (bis(diethylamino)methylene)phosphoramidofluoridate, methyl (E)-(1-(diethylamino)ethylidene)phosphoramidofluoridate, N-(bis(diethylamino)methylene)-P-methylphosphonamidic fluoride, O-(3,3-dimethylbutan-2-yl) methylphosphonofluoridoselenoate, O-isopropyl methylphosphonofluoridoselenoate, 0-pentyl methylphosphonofluoridoselenoate, phenyl N-(bis(dimethylamino)methylene)-P-methylphosphonamidate, or S-(2-(diethylamino)ethyl) O-isobutyl methylphosphonothioate.

In some embodiments, the disclosure is directed to a method of counteracting the effects of a carbamate nerve agent in a patient in need thereof, comprising administering to the skin of said patient an effective amount of a composition of the present disclosure. In some embodiments, the carbamate nerve agent is N,N-diethyl-N-methyl-3-[(methylcarbamoyl)oxy]anilinium chloride, 1,8-bis[methyl-2(3-dimethylcarbamoxypyridyl)methylamino]octane dimethobromide, or 1,9-bis[methyl-2(3-dimethylcarbamoxypyridyl)methylamino]nonane dimethobromide.

The following example is provided to illustrate the compositions, processes, and properties of the present disclosure. The example is merely illustrative and not intended to limit the disclosure to the materials, conditions, or process parameters set forth therein.

### EXAMPLE

### Example 1. Aqueous Composition for Transdermal Administration of an Antidote to a Poison

Nonaethylene glycol monododecyl ether (3 mL), 1-methyl-2-pyrrolidinone (0.3 mL), ethanol (4 mL), oleic acid (1 mL), and water (50 mL) are combined to form an admixture. An effective amount of a poison antidote is combined with the admixture to form a transdermal composition. The transdermal composition is applied to the skin of a patient in an amount and for a time sufficient for the poison antidote to permeate through the skin and into the patient's bloodstream to counteract the effect of a poison.

### Example 2. Aqueous Composition for Transdermal Administration of Atropine Sulfate

Nonaethylene glycol monododecyl ether (3 mL), 1-methyl-2-pyrrolidinone (0.3 mL), ethanol (4 mL), oleic acid (1 mL), and water (50 mL) are combined to form an admixture. An effective amount of atropine sulfate is combined with the admixture to form a transdermal composition. The transdermal composition is applied to the skin of a patient in an amount and for a time sufficient for the poison antidote to permeate through the skin and into the patient's bloodstream to counteract the effect of a poison.

### Example 3. Aqueous Composition for Transdermal Administration of Pralidoxime Chloride

Nonaethylene glycol monododecyl ether (3 mL), 1-methyl-2-pyrrolidinone (0.3 mL), ethanol (4 mL), oleic acid (1 mL), and water (50 mL) are combined to form an admixture. An effective amount of pralidoxime chloride is combined with the admixture to form a transdermal composition. The transdermal composition is applied to the skin of a patient in an amount and for a time sufficient for the poison antidote to permeate through the skin and into the patient's bloodstream to counteract the effect of a poison.

### Example 4. Aqueous Composition for Transdermal Administration of a Combination of Atropine Sulfate and Pralidoxime Chloride

Nonaethylene glycol monododecyl ether (3 mL), 1-methyl-2-pyrrolidinone (0.3 mL), ethanol (4 mL), oleic acid (1 mL), and water (50 mL) are combined to form an admixture. Effective amounts of aldoxime sulfate and pralidoxime chloride are combined with the admixture to form a transdermal composition. The transdermal composition is applied to the skin of a patient in an amount and for a time sufficient for the poison antidote to permeate through the skin and into the patient's bloodstream to counteract the effect of a poison.

### Example 5. Anhydrous Composition for Transdermal Administration of an Antidote to a Poison

Nonaethylene glycol monododecyl ether (3 mL), 1-methyl-2-pyrrolidinone (0.3 mL), ethanol (4 mL), and linoleic acid (1 mL) are combined to form an admixture. An effective amount of a poison antidote is combined with the admixture to form a transdermal composition.

### Example 5. Anhydrous Composition for Transdermal Administration of Atropine Sulfate

Nonaethylene glycol monododecyl ether (3 mL), 1-methyl-2-pyrrolidinone (0.3 mL), ethanol (4 mL), and linoleic acid (1 mL) are combined to form an admixture. An effective amount of atropine sulfate is combined with the admixture to form a transdermal composition.

### Example 6. Anhydrous Composition for Transdermal Administration of Pralidoxime Chloride

Nonaethylene glycol monododecyl ether (3 mL), 1-methyl-2-pyrrolidinone (0.3 mL), ethanol (4 mL), and linoleic acid (1 mL) are combined to form an admixture. An effective amount of pralidoxime chloride is combined with the admixture to form a transdermal composition.

### Example 7. Anhydrous Composition for Transdermal Administration of a Combination of Atropine Sulfate and Pralidoxime Chloride

Nonaethylene glycol monododecyl ether (3 mL), 1-methyl-2-pyrrolidinone (0.3 mL), ethanol (4 mL), and linoleic acid (1 mL) are combined to form an admixture. An effective amount of atropine sulfate is combined with the admixture to form a transdermal composition.

### Example 8. Transdermal Administration of an Antidote Using an Aqueous Composition

The transdermal composition of Example 1 is applied to the skin of a patient in an amount and for a time sufficient for the poison antidote to permeate through the skin and into the patient's bloodstream to achieve a therapeutic effect.

### Example 9. Transdermal Administration of an Antidote Using an Anhydrous Composition

The transdermal composition of Example 5 is applied to the skin of a patient in an amount and for a time sufficient for the poison antidote to permeate through the skin and into the patient's bloodstream to achieve a therapeutic effect.

### Example 10. Transdermal Administration of an Antidote to Counteract a G-Series Nerve Agent

The transdermal composition of Example 1 is applied to the skin of a patient in an amount and for a time sufficient for the poison antidote to permeate through the skin and into the patient's bloodstream to counteract the effects of a G-series nerve agent.

### Example 11. Transdermal Administration of an Antidote to Counteract a V-Series Nerve Agent

The transdermal composition of Example 1 is applied to the skin of a patient in an amount and for a time sufficient for the poison antidote to permeate through the skin and into the patient's bloodstream to counteract the effects of a V-series nerve agent.

### Example 12. Transdermal Administration of an Antidote to Counteract a Novichok Nerve Agent

The transdermal composition of Example 1 is applied to the skin of a patient in an amount and for a time sufficient for the poison antidote to permeate through the skin and into the patient's bloodstream to counteract the effects of a Novichok nerve agent.

What is claimed:

1. A composition comprising:
i) a poison antidote;
ii) a second component comprising:
(1) a compound of formula I
R—(OCH₂CH₂)_{y}—OH (I)
wherein R is C₁₋₂₀alkyl, C₂₋₂₀alkenyl; or C₂₋₂₀alkynyl; and y is 1 to 25;
(2) a tetrafunctional block copolymer surfactant terminating in primary hydroxyl groups;
(3) a sorbitan derivative;
(4) a C₈₋₁₀alkyl ammonium salt;
(5) a compound of formula II
HO—(CH₂CH₂O)ₘ—C(CH₃)(C₄H₉)—C≡C—C(CH₃)(C₄H₉)—(OCH₂CH₂)ₙ—OH (II)
wherein m and n are each independently 1 to 25; or
(6) a combination thereof;
iii) a third component comprising:
(1) an amide of the formula III
R²—N(R¹)—C(O)—R³ (III)
wherein each R¹ is independently H or C₁₋₃alkyl; and R² and R³ are independently C₁₋₇alkyl or together with the atoms to which they are attached, form a lactam having 3 to 10 carbon atoms;
(2) a sulfoxide;
(3) a urea;
(4) ethyl acetate; or
(5) a combination thereof;
iv) a C₂₋₁₀ alkyl alcohol;
v) an organic acid having 1 to 25 carbon atoms; and
vi) optionally, water.

2. The composition of claim 1, wherein said poison antidote is acetylcysteine, acetylcholinesterase, biperiden, butyrylcholinesterase, atropine, dimercaprol, flumazenil, amyl nitrite, sodium nitrite/sodium thiosulfate, hydroxocobalamin, fomepizole, leucovorin, EDTA, deferoxamine, a pralidoxime salt, nalmefene, potassium 2,3-butanedione monoximate, 1-[[[4-(aminocarbonyl)-pyridinio]-methoxy]-methyl]-2-[(hydroxyimino) methyl] pyridinium dichloride, 2-[(hydroxyimino)methyl]-1-[4-(tert-butyl)benzyl] pyridinium bromide, or quaternary ammonium salts thereof, or pharmaceutically acceptable salts thereof, or mixtures thereof.

3. The composition of claim 2 wherein the poison antidote is atropine or a pharmaceutically acceptable salt thereof.

4. The composition of claim 2 wherein the poison antidote is a pralidoxime salt.

5. The composition of claim 4 wherein the pralidoxime salt is pralidoxime chloride, pralidoxime bromide, or pralidoxime iodide.

6. The composition of claim 2 wherein the poison antidote is a mixture of atropine or pharmaceutically acceptable salt thereof; and a pralidoxime salt.

7. The composition of claim 6 wherein the pralidoxime salt is pralidoxime chloride, pralidoxime bromide, or pralidoxime iodide.

8. The composition of claim 1 wherein said second component comprises a compound of formula I.

9. The composition of claim 8, wherein R is C₁₋₂₀alkyl.

10. The composition of claim 8, wherein y is 5 to 15.

11. The composition of claim 8, wherein said compound of formula I is cetomacrogol 1000; octadecan-1-ol, ethoxylated; polyoxyethylene(12)tridecyl ether; polyoxyethylene(10)tridecyl ether; fatty alcohol polyoxyethylene ether, polyoxyethylene branched nonylcyclohexyl ether, nonaethylene glycol monododecyl ether, 23-{[4-(2,4,4-trimethyl-2-pentanyl)cyclohexyl]oxy}-3,6,9,12,15,18,21-heptaoxatricosan-1-ol, or a combination thereof.

12. The composition of claim 8, wherein R is C₂₋₂₀alkenyl.

13. The composition of claim 8, wherein the compound of formula I is polyoxyl(10)oleyl ether, polyethylene glycol tert-octylphenyl ether, or a combination thereof.

14. The composition of claim 8, wherein R is C₂₋₂₀alkynyl.

15. The composition claim 1, wherein said second component comprises a tetrafunctional block copolymer surfactant terminating in primary hydroxyl groups.

16. The composition of claim 15, wherein said tetrafunctional block copolymer surfactant terminating in primary hydroxyl groups is ethylenediaminetetrakis(ethoxylate-Block-propoxylate).

17. The composition of claim 1, wherein said second component comprises a sorbitan derivative.

18. The composition of claim 17, wherein the sorbitan derivative is polyoxyethylene sorbitan tetraoleate, 1,4-anhydro-6-O-palmitoyl-D-glucitol (sorbitan, monohexadecanoate), a polyethylene glycol sorbitan monolaurate, or a combination thereof.

19. The composition of claim 1, wherein said second component comprises a C₈₋₁₀alkyl ammonium salt.

20. The composition of claim 19, wherein said C₈₋₁₀alkyl ammonium salt is methyltrialkyl(C₈-C₁₀)ammonium chloride (ADOGEN 464).

21. The composition of claim 1, wherein said second component comprises a compound of formula II.

22. The composition of claim 1 wherein said third component comprises a compound of formula III.

23. The composition of claim 22, wherein R¹ is methyl, ethyl, or propyl.

24. The composition of claim 22, wherein R² and R³, together with the atoms to which they are attached, form a lactam having 3 to 10 carbon atoms.

25. The composition of claim 24, wherein the lactam is a pyrrolidone.

26. The composition of claim 1 wherein the third component comprises a sulfoxide.

27. The composition of claim 1 wherein the third component comprises a urea.

28. The composition of claim 1 wherein the third component comprises ethyl acetate

29. The composition of claim 1 wherein the C₂₋₁₀alkyl alcohol is glycerol, propylene glycol, ethanol, isopropanol, 1-propanol, butanol, t-butanol, pentanol, 1-octanol, or a combination thereof.

30. The composition of claim 1 wherein the organic acid is a fatty acid or a C₁₋₆alkyl acid.

31. The composition of claim 1 in the form of a gel, transdermal patch, lotion, cream, spray, emulsion, or dispersion.

32. A method comprising applying a composition of claim 1 to the skin of a mammal for a time sufficient to achieve permeation of at least a portion of said poison antidote through the skin.

33. A method comprising administering a composition of claim 1 to the skin of a mammal for a time and under conditions effective to achieve passage of at least a portion of said composition through said skin.

34. A method of counteracting the effects of a poison in a mammal in need thereof, comprising administering to the skin of said mammal an effective amount of a composition of claim 1.

35. The method of claim 34, wherein said poison is a nerve agent.

36. The method of claim 35, wherein said nerve agent is a G-series nerve agent.

37. The method of claim 36 wherein said G-series nerve agent is ethyl N,N-dimethylphosphoramidocyanidate (tabun, GA), propan-2-yl methylphosphonofluoridate (sarin, GB), O-pinacolyl methylphosphonofluoridate (soman, GD), cyclohexyl methylphosphonofluoridate (cyclosarin, GF), or 2-(Dimethylamino)ethyl N,N-dimethylphosphoramidofluoridate) (GV).

38. The method of claim 35, wherein said nerve agent is a V-series nerve agent.

39. The method of claim 38 wherein said V-series nerve agent is (S)-(ethyl {[2-(diethylamino)ethyl]sulfanyl}(ethyl)phosphinate) (VE), O,O-Diethyl S-[2-(diethylamino)ethyl] phosphorothioate (VG), S-[2-(Diethylamino)ethyl] O-ethyl methylphosphonothioate (VM), N,N-diethyl-2-(methyl-(2-methylpropoxy)phosphoryl)sulfanylethanamine (VR), Ethyl ({2-[bis(propan-2-yl)amino]ethyl}sulfanyl)(methyl)phosphinate (VX), or O-cyclopentyl S-(2-diethylaminoethyl) methylphosphonothiolate (EA-3148).

40. The method of claim 35, wherein said nerve agent is a Novichok agent.

41. The method of claim 40 wherein said Novichok agent is ([(2-chloro-1-methylpropoxy)fluorohydroxyphosphinyl]oxy)carbonimidic chloride fluoride, (E)-N-(1-(diethylamino)ethylidene)-P-methylphosphonamidic fluoride, 1-chloropropan-2-yl(E)-(((chlorofluoromethylene)amino)oxy)phosphonofluoridate, 2-(dimethylamino)ethyl ethyl dimethylphosphoramidate, ethyl (bis(diethylamino)methylene)phosphoramidofluoridate, ethyl (E)-(1-(diethylamino)ethylidene)phosphoramidofluoridate, ethyl dimethylphosphoramidofluoridate, ethyl N-[(1E)-1-(diethylamino)ethylidene]-phosphoramidofluoridate, methyl (bis(diethylamino)methylene)phosphoramidofluoridate, methyl (E)-(1-(diethylamino)ethylidene)phosphoramidofluoridate, N-(bis(diethylamino)methylene)-P-methylphosphonamidic fluoride, O-(3,3-dimethylbutan-2-yl) methylphosphonofluoridoselenoate, O-isopropyl methylphosphonofluoridoselenoate, O-pentyl methylphosphonofluoridoselenoate, phenyl N-(bis(dimethylamino)methylene)-P-methylphosphonamidate, or S-(2-(diethylamino)ethyl) O-isobutyl methylphosphonothioate.

42. The method of claim 35, wherein said nerve agent is a carbamate.

43. The method of claim 42, wherein the nerve agent is N,N-diethyl-N-methyl-3-[(methylcarbamoyl)oxy]anilinium chloride, 1,8-bis[methyl-2(3-dimethylcarbamoxypyridyl)methylamino]octane dimethobromide, or 1,9-bis[methyl-2(3-dimethylcarbamoxypyridyl)methylamino]nonane dimethobromide.

44. The method of claim 34, wherein said mammal is a human being.
